(19) United States
(12) Patent Application Publication
(10) Pub. No.: US 2007/0169778 A1
(43) Pub. Date: Jul. 26, 2007

(54) Device for pulmonary drug delivery

(76) Inventors: Peter Richard Smith, Leicestcrshire (GB); Olga Kusmartseva, Leicestershire (GB)

Correspondence Address: HARRINGTON & SMITH, PC, 4 RESEARCH DRIVE, SHELTON, CT, 06484-6212, US

(21) Appl. No.: 10/553,364

(22) PCT Filed: Apr. 16, 2004
(86) PCT No.: PCT/GB04/01714

§ 371 Date: Sep. 21, 2006

(30) Foreign Application Priority Data
Apr. 16, 2003 (GB) 0308771.5

(51) Int. Cl. A61M 16/00; A61M 15/00; B65D 83/06

(52) U.S. Cl. 128/203.25; 128/203.12; 128/203.15

(57) ABSTRACT

A method of assessing the effectiveness of pulmonary drug delivery, including the steps of: a) providing a drug into an air flow past a sensor including a radiation source and a radiation detector; b) detecting, at the radiation detector, incident radiation over a period of time as a measurement profile; c) quantifying at least one characteristic of the shape of a measurement profile; and d) producing an indication of the effectiveness of pulmonary drug delivery based upon the at least one quantified characteristic.

Embodiments of the present invention relate to pulmonary drug delivery. It particular they relate to apparatus and methods for the assessment of the effectiveness of pulmonary drug delivery.

The assessment of the effectiveness of a drug for pulmonary delivery is currently carried out in the laboratory using a twin stage impinger (TSI) apparatus. This apparatus draws the drug through a convoluted passage using a vacuum pump at a high flow rate for a reasonably long period e.g. 60 l/min for 5 seconds. The convoluted passage has a first stage at a first sharp bend for capturing large drug particles in liquid, and a second stage for capturing fine drug particles in liquid. The liquid at the first stage is analysed to determine the mass of large particles of the drug captured there. The liquid at the second stage is analysed to determine the mass of fine particles of the drug captured there.

The effectiveness of a pulmonary drug depends upon its fine particle mass. This represents the amount of drug which is of the correct size (e.g. 0.5 to 6 μm) to reach deep within the lung and have a desirable physiological effect on a user. The drug particles that are greater in size than fine particles tend to be absorbed into a user's digestion system, which may cause side effects. The total mass of drug delivered when compared to the fine particle mass, indicates the efficiency of the drug delivery to the lung. When it is expressed as the ratio of the fine particle mass to the total dose mass, it is referred to as the fine particle fraction.

There are several disadvantages associated with the TSI procedure.

It is a difficult and time intensive procedure and may take a day to complete a single assessment. It may therefore take weeks or months to obtain enough data to determine statistical variance of the drug delivery process.

Another disadvantage is that the apparatus does not necessarily give results that are representative of actual drug delivery in vivo. The apparatus uses an air flow rate (e.g. 60 l/min) that is not necessarily representative of particular human's breath in-take and for a period of time (5 s) longer than a normal breath in-take.

Another disadvantage is that the apparatus tests the delivery properties of the drug independently of the user for whom the drug is intended.

It would be desirable to provide an improved assessment procedure.

According to a first aspect of the present invention there is provided a method of assessing the effectiveness of pulmonary drug delivery, comprising the steps of: a) providing a drug into an air flow past a sensor comprising a radiation source and a radiation detector; b) detecting, at the radiation detector, incident radiation over a period of time as a measurement profile; c) quantifying at least one characteristic of the shape of a measurement profile; and d) producing an indication of the effectiveness of pulmonary drug delivery based upon the at least one quantified characteristic.

The indication indicates how successful the drug delivery was i.e. the degree of success, and not whether drug delivery did or did not occur. It is typically a quantitative measure of the effectiveness of drug delivery.

There is also provided a measurement device for assessing the effectiveness of pulmonary drug delivery, comprising: a conduit through which air carrying a cloud of drug particles can flow during drug delivery; a radiation source for providing radiation into the conduit; a radiation detector for detecting radiation from the conduit over a period of time as a measurement profile; and a processor operable to quantify one or more characteristics of the shape of a measurement profile and to produce an indication of the effectiveness of pulmonary drug delivery based upon the quantified characteristic(s).

According to a second aspect of the invention there is provided a method of assessing the effectiveness of pulmonary drug delivery, comprising the steps of: recording, during a drug delivery, the output of a first radiation detector against time as a first measurement profile; recording, during the same drug delivery, the output of a second radiation detector against time as a second measurement profile; and processing the first and second measurement profiles to produce an indication of the effectiveness of pulmonary drug delivery.

There is also provided a measurement device for assessing the effectiveness of pulmonary drug delivery, comprising: a conduit through which air carrying a cloud of drug particles can flow during drug delivery; a radiation source for providing radiation into the conduit; a first radiation detector for detecting radiation from the conduit over a period of time as a first measurement profile; a second radiation detector for detecting radiation from the conduit over the period of time as a second measurement profile; and a processor operable to produce an indication of the effectiveness of pulmonary drug delivery based upon the first and second measurement profiles.

Embodiments of these aspects of the invention consequently provide a faster assessment procedure. This allows information on the statistical variance of the effectiveness of pulmonary drug delivery to be produced.

The air flow may be created by a person or a breathing simulator. Embodiments of the invention consequently provide an assessment procedure that is representative of in vivo drug delivery and can take into account the person for whom the drugs are intended.

The measurement device can be attached to or integrated within an actual drug delivery device. Embodiments of the invention consequently provide an assessment procedure that takes into account the device used in situ for drug delivery.

According to a third aspect of the present invention there is provided a drug delivery device for providing a drug dose to a user in a plurality of separate drug deliveries, comprising: a drug metering means for releasing a controlled amount of drug for each drug delivery; a conduit through which air carrying a cloud of drug particles can flow; a radiation source for providing radiation into the conduit; a first radiation detector for detecting radiation from the conduit during a on-going drug delivery as a first measurement profile; and control means operable to control the drug metering means, for a subsequent drug delivery, in dependence upon at least the first measurement profile.

For a better understanding of the present invention reference will now be made by way of example only to the accompanying drawings in which:

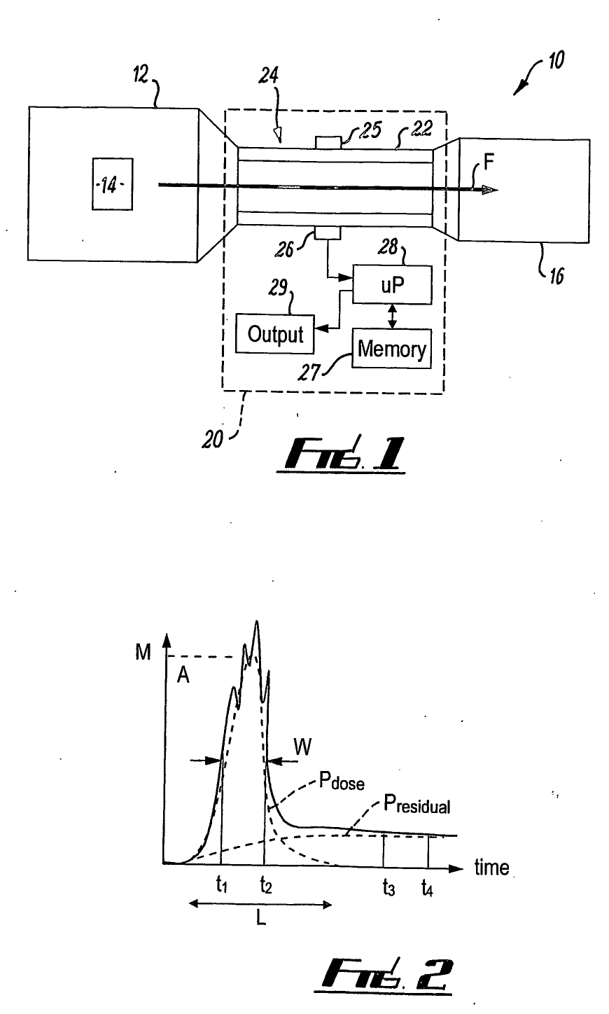

FIG. 1 illustrates an assessment system for the rapid assessment of pulmonary drug delivery;

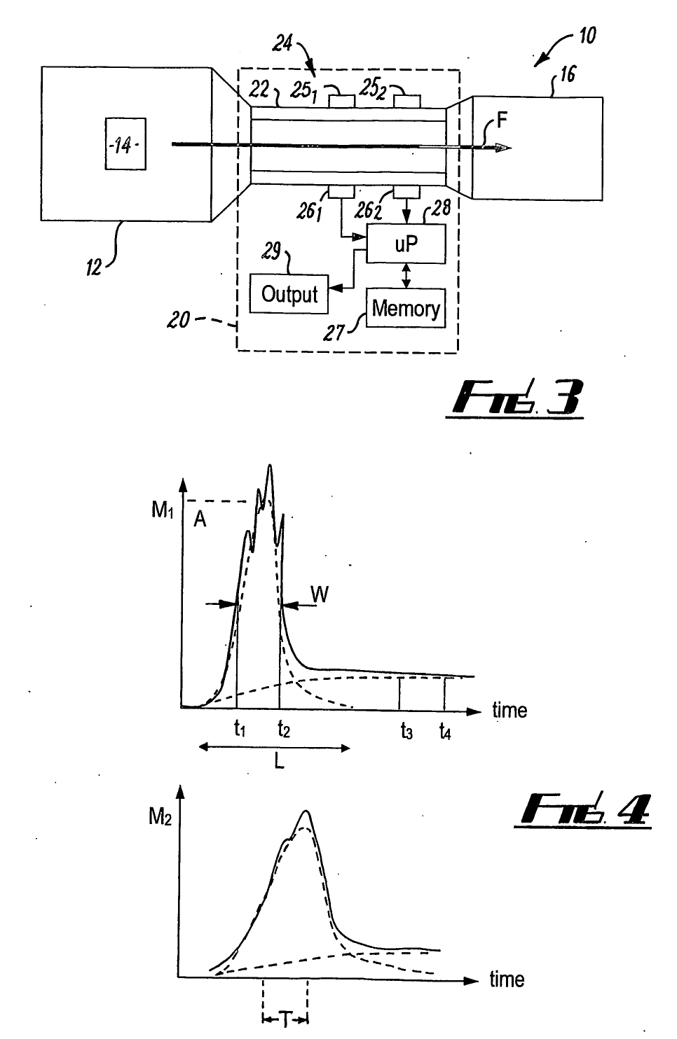

FIG. 2 illustrates a typical measurement profile;

FIG. 3 illustrates an alternative embodiment of the assessment system;

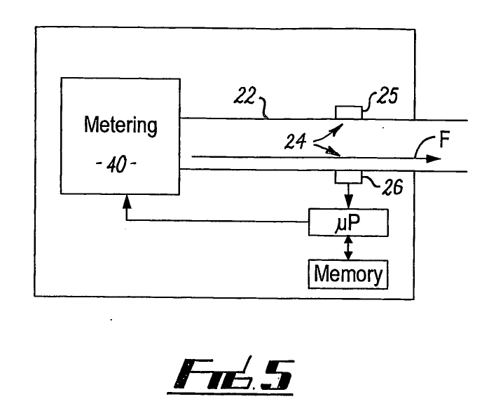

FIG. 4 illustrates a first measurement profile M1 and a second measurement profile M2 for a single drug delivery; and

FIG. 5 illustrates an adaptive-multi-dose drug delivery device.

FIG. 1 illustrates an assessment system 10 for the rapid assessment of in vivo pulmonary drug delivery. The system 10 comprises in axial flow series a drug delivery device 12 including drug 14 for pulmonary delivery, a measurement device 20 and a physiological actuator 16. A flow of air F is drawn by the physiological actuator 16 from the drug delivery device 12, through the measurement device 20. A seal may be required at the interface between the drug delivery device 12 and the measurement device 20 and a seal may be required between the physiological actuator 16 and the measurement device 20.

The air flow F created by the physiological actuator 16, may aerosolises drug agglomerates in the air flow F and create a cloud of drug particles or the air flow F may draws an already existing aerosol cloud into the lung. The size of the particles and the distribution of particles within the cloud change as the cloud moves in the air flow F.

The effectiveness of a pulmonary drug depends upon its fine particle mass. This represents the amount of drug which is of the correct size (e.g. 0.5 to 6 μm) to reach deep within the lung and have a desirable physiological effect on a user. The drug particles that are greater in size than fine particles tend to be absorbed into a user's digestion system, which may cause side effects. The total mass of drug delivered when compared to the fine particle mass, indicates the efficiency of the drug delivery to the lung. When it is expressed as the ratio of the fine particle mass to the total dose mass, it is referred to as the fine particle fraction.

Aerosolised particle clouds scatter and absorb radiation according to the cloud composition, particularly the particle concentration and particle size distribution within the cloud. The system 10 is arranged to quantitatively assess the effectiveness of pulmonary drug delivery from a measurement profile that indicates how detected radiation varies as the drug cloud passes between a radiation source and a radiation detector.

The drug for pulmonary delivery may be in any formulation including dry or liquid form or formulated as a solution/suspension with a solvent.

The drug delivery device 12 is a real pulmonary drug delivery device. It could be a currently marketed device or a new design of device intended for the market. Examples of the possible types of suitable pulmonary drug delivery devices include: metered dose inhalers, dry powder inhalers, nebulizers, single breath liquid systems, and metered solution inhalers.

The physiological actuator may be provided by a breath in-take of a person or by the operation of a breathing simulator.

The measuring device 20 includes a straight optically translucent tube 22 connected between the output of the drug delivery device 12 and the physiological actuator 16. The tube 22, in this example, has a 21 mm internal diameter and a fixed length of 60 mm. In other embodiments the tube 22 may have an internal diameter up to 30 mm and a fixed length of between 5 and 200 mm.

The measuring device also comprises a sensor 24 that is exterior to the tube 22, a processor connected to the sensor 24, a memory 27 and an output 29.

The sensor 24 includes a radiation source 25 and a radiation detector 26 lying in a plane perpendicular to the longitudinal axis of the tube 22 and the flow direction F. In this example, the sensor 24 operates by obscuration of light and the light source 25 and light detector 26 are positioned diametrically opposite each other. In other embodiments, the sensor 24 operates by light scattering and the source and detector are positioned in the same plane but the detector is not positioned in the ‘line-of-sight’ of the light source so that it detects light at a predetermined scattering angle.

The processor 28 is programmed to record, during a drug in-take from the drug delivery device 12 by the physiological actuator 16, real-time data from the sensor 24 in the memory 27. The real-time data is a measurement profile of how the detected radiation varies with time.

The processor 28 may start recording data in response to user input. For example, a button of a user interface of the measurement device 20 could be depressed to start recording. Alternatively, the processor 28 could start recording automatically in response to a detection of the start of the in-take procedure. For example, a flow detector could be positioned upstream of the sensor 24, and the processor 28 could detect when the detected flow rate exceeds a predetermined threshold.

A typical measurement profile is illustrated in FIG. 2. It records how the output M of the detector 26 varies with time as a drug cloud passes between the source 25 and detector 26. The inventors have determined how the shape of the measurement profile is sensitive to particle concentration and particle size distribution within the drug cloud.

The processor 28 is programmed to automatically process, in situ, the recorded measurement profile to assess the pulmonary drug delivery from the drug in-take in real-time. The processor 28 quantifies characteristics of the shape of the measurement profile and produces a quantified indication of, for example, the dose delivered, the fine particle dose delivered and the fine particle fraction delivered based upon the quantified characteristics. The results of the processing are provided to output 29, which could for example be a display.

The processing of the measurement profile starts with the fitting of a mathematical function P to the measurement profile. The function P is the sum of two parts: a dose function P_{dose} and a level transition ( [symbol] ) residual function P_{residual}.

Quantitative values of parameters characterising the shape of the measurement profile are extracted from the fitted dose function P_{dose} and from the fitted residual function P_{residual}.

The characteristic parameters may include:

a) The width W of the dose function P_{dose}, for example, the full width at half maximum. This is a time.

b) The maximum amplitude A of the dose function P_{dose},

c) The length L of the dose function P_{dose}

d) The asymmetry of the dose function P_{dose}

e) The deviation of the actual measurement profile from the fitted mathematical function P

f) The height H of the residual function P_{residual}

It has been determined by the inventors that the value of the maximum amplitude A of the dose function P_{dose} is correlated to the fine particle mass of the measured pulmonary drug cloud. Therefore, the quantitative value of the maximum amplitude A gives a quantitative indication of the fine particle mass in non-SI units. The memory 27 may store calibration data, which allows the processor 28 to convert the quantitative indication to a mass value in SI units.

It has been determined by the inventors that the value of the dose function P_{dose}, integrated over the width W is correlated to the total dose mass of the measured pulmonary drug cloud. Therefore, the quantitative value of the integral gives a quantitative indication of the dose mass in non-SI units. The memory 27 may store calibration data, which allows the processor 28 to convert the quantitative indication to a mass value in SI units.

It has been determined by the inventors that the value of the maximum amplitude A of the dose function P_{dose} divided by the value of the dose function P_{dose} integrated over the width W, is correlated to the fine particle fraction of the measured pulmonary drug cloud. Therefore, the quantitative value of the fraction gives a quantitative indication of the fine particle fraction in non-SI units. The memory 27 may store calibration data that allows the processor 28 to convert the quantitative indication to standard units.

The inventors have also determined that the length L is correlated to the pulmonary drug cloud volume and length, the asymmetry of the dose function P_{dose} is correlated to drug delivery cloud asymmetry, the deviation of the measurement profile from the fitted function P is correlated to the cloud homogeneity and that the height H of the residual function P_{residual} is correlated to the drug dose that is lost by adhering to the side walls of the passage through which the drug is delivered.

Thus, the quantitative values of parameters characterising the shape of the measurement profile extracted from the fitted curve P, the fitted dose function P_{dose} and from the fitted residual function P_{residual} may be used by the processor 28 to provide quantitative indications of the efficiency of the drug delivery process and/or of the drug delivery cloud.

The system 10 can be used to easily repeat an assessment procedure and then determine the statistical variation between the results of the repeated procedures. The simple, reliable and robust technology allows an assessment to be completed quickly and for a statistically significant number of assessments to be completed in a short period of time (hours).

The processor 28 may be programmed to quantify the variation in the quantitative indications of the efficiency of the drug delivery process and/or of the drug delivery cloud. For example, the processor 28 may store the determined fine particle fraction for each drug delivery assessment in the memory 27. After a number of assessments, the processor 28 can perform statistical analysis on the sample of fine particle fractions stored. It may, for example, provide the mean fine particle fraction and the standard deviation from the mean. Alternatively, the processor 28 may store the separate measurement profiles or characteristic parameters for each assessment and average them before using the average to provide indications of the efficiency of the drug delivery process and/or of the drug delivery cloud.

The system 10 comprises three distinct components which are very important to the drug delivery process: the drug delivery device 12, the pulmonary drug formulation 14 and the physiological actuator 16.

The system 10 allows the efficiency of a new drug delivery device to be assessed by controlling the physiological actuation by using a breathing simulator and by using a sample of material with known properties as the drug formulation 14.

The system 10 allows the efficiency of a new drug formulation to be assessed by controlling the physiological actuation by using a breathing simulator and by using a standard drug delivery device 12.

The system 10 allows the assessment of self-administration of a pulmonary drug 14 by a person using a drug delivery device 12. The device may indicate whether a user needs to inhale harder or softer. The system may be used with a placebo drug to train a person how to use a pulmonary drug delivery device.

FIG. 3 illustrates an alternative embodiment of the assessment system 10. Where the same reference numerals are shared with FIG. 2 they indicate the same components. The system 10 has a different measurement device 20.

The measurement device 20 has a plurality of sensors 24₁, 24₂ . . . In this example, two sensors are illustrated but more than two sensors may be used.

A first sensor 24₁, includes a first radiation source 25₁ and a first radiation detector 26₁ lying in a first plane perpendicular to the longitudinal axis of the tube 22 and the flow direction F. The first plane is located at position x₁ along the longitudinal axis of the tube 22.

A second sensor 24₂, includes a second radiation source 25₂ and a second radiation detector 26₂ lying in a second plane perpendicular to the longitudinal axis of the tube 22 and the flow direction F. The second plane is located at position x₂ along the longitudinal axis of the tube 22.

In this example, the sensors 24ₙ operate by obscuration of light and the light source 25ₙ and light detector 26ₙ are positioned diametrically opposite each other. In other embodiments, the sensors 24ₙ operate by light scattering and while the source and detector are still positioned in the same plane, the detector is not positioned in the ‘line-of-sight’ of the light source so that it detects light at a predetermined scattering angle.

The processor 28 records in memory 27 a first measurement profile from the first sensor 24₁ and a second measurement profile from the second sensor 24₂ during a drug delivery. An illustrative first measurement profile M1 and a second measurement profile M2 are shown in FIG. 4. The processor 28 independently processes the first measurement profile M1 and the second measurement profile M2 as described above to produce a first set of quantitative values for characteristic parameters from the first measurement profile and a second set of quantitative values for characteristic parameters from the second measurement profile.

The quantitative values of the first set of parameters characterising the shape of the first measurement profile provide quantitative indications of the efficiency of the drug delivery process and/or status of the drug delivery cloud at position x₁.

The quantitative values of the second set of parameters characterising the shape of the second measurement profile provide quantitative indications of the efficiency of the drug delivery process and/or status of the drug delivery cloud at position x₂.

A comparison of the first measurement profile or results obtained from the first measurement profile with the second measurement profile or the results obtained from the second measurement profile provide information on the dynamics of the drug cloud. For example, the evaporation of propellant in a liquid delivery system may result in an increase in the maximum amplitude from the first measurement to the second measurement response.

The processor 28 is arranged to cross-correlate the first measurement profile with the second measurement profile to obtain a time off-set T between the profiles. The dose functions P_{dose} may be cross correlated instead of the measurement profiles. The distance between x₁ and x₂ is stored in memory 27 and is divided by the time off-set T by the processor 28, to obtain an indication of the average speed of the drug cloud through the tube 22. The speed of the drug cloud gives an indication of the percentage of the drug cloud that will be deposited on the back of the throat. The processor 28 is arranged to provide the indication of the average speed via the output 29.

Another alternative embodiment of the assessment system 10 uses one or more sensors that detect light at different frequencies. The pulmonary drug 14 for delivery includes pharmacologically inactive large carrier particles (e.g. lactose) coloured with a first colour and coated with a pharmacologically active drug coloured a second colour. The drug is designed to leave the carrier in transit. As it does so the proportion of the second colour detected increases and the fine particle fraction for the second colour increases. The effectiveness of the drug release from the carrier can therefore be determined.

Although the measurement device 20 has been described as a separate add-on component to the drug delivery device 12, in other embodiments the functionality of the measurement device 20 may be integrated into the drug delivery device 12.

FIG. 5 illustrates a multi-dose drug delivery device 12. A user of the device self administers the correct dose by performing a plurality of inhalations using the device. Each inhalation causes a drug delivery to the user. The device automatically varies the amount of drug delivered in each drug delivery and/or the number of drug deliveries required. This is particularly useful if the user has little or variable wind and cannot inhale forcefully or consistently.

The functionality of the measurement device 20 is integrated into the drug delivery device 12.

The device 12 has a metering unit 40 which meters the amount of drug that is available for drug delivery on inhalation. The metering unit 40 receives an input from the processor 28.

On a first inhalation, a predetermined amount of drug is released by the metering unit 40. The sensor 24 detects the variation in radiation detected by the radiation detector 26 as the inhaled drug cloud passes between the radiation source 25 and radiation detector 26. The processor 28 records the measurement profile in the memory 27 and then processes the measurement profile as described above to determine the fine particle fraction and/or dose of the inhaled first dose.

The processor then controls the amount of drug released by the metering unit 40 for the subsequent drug delivery on the next inhalation. Alternatively, or in addition, the processor may determine whether and how many additional drug delivery inhalations are required. An adaptive feedback system is thus created that controls, in dependence upon the effectiveness of the drug delivery in the preceding inhalation, the amount of drug to be released in a subsequent inhalation or inhalations. The effectiveness of the drug delivery in the preceding inhalation may be determined from the characteristic(s) of a detected measurement profile when a single detector is used or from a comparison of the measurement profiles from separate detectors when two or more detectors are used.

Although embodiments of the present invention have been described in the preceding paragraphs with reference to various examples, it should be appreciated that modifications to the examples given can be made without departing from the scope of the invention as claimed.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1-54. (canceled)

55. A method of assessing the effectiveness of pulmonary drug delivery, comprising the steps of:
a) providing a drug into an air flow past a sensor comprising a radiation source and a radiation detector;
b) detecting, at the radiation detector, incident radiation over a period of time as a measurement profile;
c) quantifying at least one characteristic of the shape of a measurement profile; and
d) producing an indication of the effectiveness of pulmonary drug delivery based upon the at least one quantified characteristic.

56. A method as claimed in claim 55 wherein the indication of the effectiveness of pulmonary drug delivery quantifies the amount of fine particles in the delivered pulmonary drug.

57. A method as claimed in claim 55, wherein the at least one characteristic is a height of the measurement profile or the height of a curve fitted to the measurement profile.

58. A method as claimed in claim 55, wherein the at least one characteristic involves an integration of the measurement profile over its width or the integration of a curve fitted to the measurement profile over its width.

59. A method as claimed in claim 57, wherein an at least one characteristic involves an integration of the measurement profile over its width or the integration of a curve fitted to the measurement profile over its width.

60. A method as claimed in claim 58, wherein the curve fitted to the measurement profile is a dose function which when summed with a level transition residual function substantially re-creates the measurement profile.

61. A method as claimed in claim 59, wherein the curve fitted to the measurement profile is a dose function which when summed with a level transition residual function substantially re-creates the measurement profile.

62. A measurement device for assessing the effectiveness of pulmonary drug delivery, comprising:
a conduit through which air carrying a cloud of drug particles can flow during drug delivery;
a radiation source for providing radiation into the conduit;
a radiation detector for detecting radiation from the conduit over a period of time as a measurement profile; and
a processor operable to quantify one or more characteristics of the shape of a measurement profile and to produce an indication of the effectiveness of pulmonary drug delivery based upon the quantified characteristic(s).

63. A measurement device as claimed in claim 62, arranged for releasable attachment to a drug dispensing device or integrated within a drug delivery device.

64. A measurement device as claimed in claim 62, wherein the indication of the effectiveness of pulmonary drug delivery indicates the fine particle component of the delivered pulmonary drug.

65. A measurement device as claimed in claim 62, wherein a quantified characteristic is the height of the measurement profile or the height of a curve fitted to the measurement profile.

66. A measurement device as claimed in claim 62, wherein a quantified characteristic involves the integration of the measurement profile over its width or the integration of a curve fitted to the measurement profile over its width.

67. A measurement device as claimed in claim 65, wherein a quantified characteristic involves the integration of the measurement profile over its width or the integration of a curve fitted to the measurement profile over its width.

68. A measurement device as claimed in claim 62 comprising a second radiation detector for detecting radiation from the conduit over a period of time as a second measurement profile, wherein the processor is operable to produce an indication of the effectiveness of pulmonary drug delivery based upon a plurality of measurement profiles for a single drug delivery.

69. A measurement device as claimed in claim 68, further comprising a second radiation source.

70. A method of assessing the effectiveness of pulmonary drug delivery, comprising the steps of:
recording, during a drug delivery, the output of a first radiation detector against time as a first measurement profile;
recording, during the same drug delivery, the output of a second radiation detector against time as a second measurement profile; and
processing the first and second measurement profiles to produce an indication of the effectiveness of pulmonary drug delivery.

71. A measurement device for assessing the effectiveness of pulmonary drug delivery, comprising:
a conduit through which air carrying a cloud of drug particles can flow during drug delivery;
a radiation source for providing radiation into the conduit;
a first radiation detector for detecting radiation from the conduit over a period of time as a first measurement profile;
a second radiation detector for detecting radiation from the conduit over the period of time as a second measurement profile; and
a processor operable to produce an indication of the effectiveness of pulmonary drug delivery based upon the first and second measurement profiles.

72. A drug delivery device for providing a drug dose to a user in a plurality of separate drug deliveries, comprising:
a drug metering means for releasing a controlled amount of drug for each drug delivery;
a conduit through which air carrying a cloud of drug particles can flow;
a radiation source for providing radiation into the conduit;
a first radiation detector for detecting radiation from the conduit during a on-going drug delivery as a first measurement profile; and
control means operable to control the drug metering means, for a subsequent drug delivery, in dependence upon at least the first measurement profile.

73. A drug delivery device as claimed in claim 72, wherein the drug metering means is arranged to vary the amount of drug released in a subsequent drug delivery, in dependence upon at least the first measurement profile or is arranged to vary the number of drug deliveries required in dependence upon at least the first measurement profile.
